(19) United States
(12) Patent Application Publication
(10) Pub. No.: US 2026/0123854 A1
(43) Pub. Date: May 7, 2026

(54) PROBE ASSEMBLY AND METHOD OF MANUFACTURE

(71) Applicant: The Regents of the University of Michigan, Ann Arbor, MI (US)

(72) Inventors: Dongxiao Yan, Ann Arbor, MI (US); Euisik Yoon, Superior Township, MI (US)

(21) Appl. No.: 19/120,671

(22) PCT Filed: Oct. 12, 2023
(86) PCT No.: PCT/US2023/035061

§ 371 Date: Apr. 11, 2025

Related U.S. Application Data
(60) Provisional application No. 63/415,778, filed on Oct. 13, 2022.

(51) Int. Cl. A61B 5/145

(52) U.S. Cl. CPC A61B 5/14503; A61B 5/14546; A61B 2562/028; A61B 2562/125; A61B 2562/164

(57) ABSTRACT

A probe assembly and method of manufacture includes probe shanks having a bend at a hinge to conform around a core region. In some embodiments, the probe shank has multiple probe sub-units which include magnets shielded from electrodes on the probe shank. Attaching the magnetized probe sub-units and/or conforming the probe shanks around a core region can include capillary induced adherence. In some embodiments, the probe assemblies and methods of manufacture overcome challenges of a monolithic fabrication process.

### FIELD

This invention relates generally to probe assemblies, and more particularly, to flexible intracortical neural probe assemblies and methods of manufacture.

### BACKGROUND

Recording capability enhancement and multifunction integration are two essential topics in neural probe development. High-channel-count neural probes have demonstrated their extraordinary strength of high spatial resolution and large tissue coverage allowing for simultaneous multi-region neural recording. Multifunctional neural probes have proven to be immensely useful for specific neuroscience applications. Miniaturized photonic components allow for localized optogenetics. Simultaneous recording of neural activities and various physiological parameters has led to new discoveries of the neurological effects of intracranial temperature, pressure, chemical concentration, and so on. Integration of a combination of functionalities, such as optical fibers and temperature sensors, drug delivery channels and chemical sensors, opens up potential for close-loop localized stimulation. The conventional approach for channel scaling and multifunctional integration is through monolithic microfabrication. However, the thin-film polymer substrate and encapsulation introduced challenges due to the limited process compatibility and adhesion issues. Increasing the number of polymer and metal layers may suffer from high process complexity, with potential contact and adhesion failure. Fabricating recording electrodes together with multiple other functional components may result in compromised performance and yield.

Expanding the field of view of a recording electrode or a neural probe can be another important aspect of recording capability enhancement, as it can increase the effective tissue coverage and the single unit yield. The conventional approach is engineering the electrodes with protruding structures, such as with the Utah array, microneedles, and fiber electrodes, or planar multi-side-exposed structures such as free-standing nanostripes and edge electrodes. For protruding structures, the electrode density and span in the depth direction can be limited. For planar, multi-side structures, the mechanical robustness during implantation and the long-term stability in physiological environments were not fully established. In addition, it can be challenging to determine the angle-of-arrival of spike signals. For certain mapping applications in which locating the neurons is critical, such electrodes may be disadvantageous.

Other self-assembly approaches were demonstrated to create cylindrical structures or 3D neural interfaces. For example, built-in mechanical stress can be used to roll composite polymer films into micro-tubular devices. However, making them suitable for brain implants would likely require sub-micro device thickness and a high stress and biocompatible actuating layer. In one approach, nanosheet electrode arrays were attached onto an optical fiber. With a more flexible and miniaturized design, it was possible to achieve a full 360 3D array. Neurotassels were created by capillary self-assembly of microelectrodes patterned at the tips of thin-film polyimide stripes. The imperfection was that the positions and orientations the electrodes were nearly random, which can make channel mapping and signal trilateration inaccurate. In comparison, the electrode array described herein is built with pre-defined geometry allowing for angular location of the neuron. While a similar capillary assisted self-assembly process could be used to create double-sided probes with 32 or 64 channels, compared with the conventional monolithically built double-sided flexible probe, the fabrication process described herein is much simpler. All the electrodes in the arrays herein can be fabricated on the same side of the substrate which is beneficial for electrode performance and yield.

All the electrodes in the arrays herein can be fabricated on the same side of the substrate which is beneficial for electrode performance and yield.

### SUMMARY

In an embodiment, there is provided a probe assembly comprising a probe shank, a hinge extending into a thickness of the probe shank, and a core region. The probe shank has a bend at the hinge to conform at least partially around the core region.

In some embodiments, the hinge extends longitudinally down the probe shank from an insertion end towards a back end.

In some embodiments, the hinge extends between a plurality of rows of electrodes.

In some embodiments, the hinge extends transversely across the probe shank from a first longitudinal side toward a second longitudinal side.

In some embodiments, the hinge delineates a conical insertion tip.

In some embodiments, the hinge has an I-shaped structure or a Z-shaped structure.

In some embodiments, the thickness of the probe shank is etched at the I-shaped structure or the Z-shaped structure.

In some embodiments, the probe shank includes a dopamine sensing electrode comprising reduced-graphene oxide on a roughened platinum-based substrate.

In some embodiments, the probe shank comprises a first probe sub-unit and a second probe-sub unit, wherein the first probe sub-unit has the hinge and the second probe-sub unit has a hinge.

In some embodiments, the first probe sub-unit and the second probe sub-unit are laminated together with magnets.

In some embodiments, the hinge comprises a plurality of fold lines that extend fully through the thickness.

In some embodiments, the core region is an optical fiber.

In some embodiments, a plurality of probe shanks are circumferentially arranged around the optical fiber, and an insertion end of each probe shank is conformally arranged around a conically shaped insertion point of the optical fiber.

In an embodiment, there is a method of manufacturing a probe assembly comprising the step of bending a portion of a probe shank around a core region at a hinge.

In some embodiments, the bending step is capillary induced.

In an embodiment, there is a probe assembly comprising a probe shank having a first probe sub-unit and a second probe sub-unit. The first probe sub-unit has a first magnet, and the second probe sub-unit has a second magnet. Either the first probe sub-unit or the second probe sub-unit has a plurality of electrodes, and the first magnet, the second magnet, or both the first magnet and the second magnet are at least partially shielded from the electrodes.

In some embodiments, each of the first magnet and the second magnet are encapsulated in polyimide.

In some embodiments, the first magnet and the second magnet are each an array of micromagnets.

In an embodiment, there is a method of manufacturing the probe assembly, comprising the step of adhering the first probe sub-unit and the second probe sub-unit with the first magnet or the second magnet.

In some embodiments, the adhering step is capillary induced.

Various aspects, embodiments, examples, features and alternatives set forth in the preceding paragraphs, in the claims, and/or in the following description and drawings may be taken independently or in any combination thereof. For example, features disclosed in connection with one embodiment are applicable to all embodiments in the absence of incompatibility of features.

### BRIEF DESCRIPTION OF THE DRAWINGS

Preferred example embodiments will hereinafter be described in conjunction with the appended drawings, wherein like designations denote like elements, and wherein:

FIG. 1 is a scanning electron microscope (SEM) image of a probe assembly, according to one embodiment;

FIG. 2 is a schematic image of a probe assembly, according to another embodiment;

FIG. 3 is a partial, exploded view of a probe shank of a probe assembly;

FIG. 4 is a schematic, cross-section view of the probe assembly of FIG. 2 that is partially assembled;

FIG. 5 is an optical microscope photo of a cross-section of a probe assembly;

FIG. 6 is a top view of hinged probe shanks according to one embodiment;

FIG. 7 is a perspective view of the embodiment of FIG. 6;

FIG. 8 is a cross-section view of the embodiment of FIGS. 6 and 7;

FIG. 9 is a top view of hinged probe shanks according to one embodiment;

FIG. 10 is a perspective view of the embodiment of FIG. 9;

FIG. 11 is a cross-section view of the embodiment of FIGS. 9 and 10;

FIG. 12 is a graph showing a comparison of lateral spring constants for various hinged and non-hinged probe shanks;

FIG. 13 is a graph showing a comparison of elastic energy for different rotating angles for various hinged and non-hinged probe shanks;

FIG. 14 schematically illustrates a method of manufacturing a hinged probe shank in accordance with one embodiment;

FIG. 15 schematically illustrates a method of manufacturing a probe shank having multiple probe sub-units in accordance with one embodiment; and

FIG. 16 schematically illustrates additional steps of the method of manufacturing shown in FIG. 15.

### DESCRIPTION OF THE ILLUSTRATED EMBODIMENTS

Described herein is a probe assembly that is constructed in accordance with a self-assembly approach that can overcome the challenges of a monolithic fabrication process. Separately fabricated individual flexible probe sub-units can serve as “building-blocks” which get laminated into a single body by magnetic and capillary hybrid self-assembly. This approach can fabricate and integrate flexible neural probes in a modularized way. Although similarly, bonding of layers fabricated on separated substrates could be achieved by using a mask aligner or manual stacking with molds, the self-assembly approach described allows for self-alignment of the probes with high accuracy (e.g., less than 20-μm longitudinal, and 5-μm transverse misalignment) and throughput with no micro-manipulation required.

In one embodiment, the probe assembly is a magnetic u-laminated intracortical interface (MULTI). MULTI has a cross-section size of 70˜130 μm wide and 8˜15 μm thick. 128 recording channels on a single 130-μm wide probe shank was achieved by laminating two probe shank layers with recording electrode arrays. Lamination of more than two probe shank layers or multi-shank probes can be achieved to further scale the channel count. Multifunctional integration of neural recording, temperature sensing and dopamine detection was achieved by laminating three probe shank layers with different components. Being able to select the desired functions and electrode array configurations allows the probe assembly to be used for various applications, allowing for high customizability and specificity.

As addressed above, creating a 3D array of electrodes by monolithic fabrication or manual assembly can be highly challenging, which can limit the total channel count and the minimum cross-section size. The disclosure herein presents a self-assembly solution to this challenge. By utilizing capillary forces, specially designed and etched thin films could be self-assembled into 3D structures by wrapping around thin optical fibers (80˜125 μm). With this approach, 3D origami probes were created that featured a total of 64 or 128 electrodes with the highest electrode density by far, with real 360° 3D recording capability. The high channel count and the insertable thin cylindrical core region makes it advantageous for acute functional mapping of the surgical trajectory with reduced tissue damage. In some embodiments, the optical fiber used for the core region further enables potential optogenetic studies.

The flexible intracortical neural probe assemblies described herein are a powerful tool for brain research for their high-spatial-resolution neural recording capability and good tissue integration. However, the conventional monolithic fabrication approach meets significant challenges when further advancing the flexible neural probe assemblies with (i) hundreds of recording channels on a single shank; (ii) integration of multiple physiological parameter sensing or stimulation components; and/or (iii) three-dimensional (3D) or double-sided electrode array configurations. Hence, the innovative self-assembly technologies and the origami neural probes of the present disclosure serve as an effective alternative to overcome these challenges. By capillary self-assembly, planar probes with special fold-lines are wrapped over thin optical fibers, which creates the high-density 3D origami probe with 64 or 128 electrodes and an 80˜105-μm diameter, for example. Angular heterogeneous single-unit signals and neural connectivity 360° surrounded the probe were identified. In another non-exclusive embodiment, by magnetic and capillary hybrid self-assembly, separately fabricated probes were laminated into one with high accuracy, which created the high-density multifunctional probe. 128 recording electrodes, a temperature sensor, and dopamine (DA) sensing electrodes were integrated on a single shank. Simultaneous multi-region neural recording and local temperature or dopamine concentration monitoring were demonstrated. Additionally, the probe longevity was validated by chronic recording with 64-channel laminated probes for up to 140 days with high signal integrity. With the technologies and probe assemblies presented, novel and highly efficient solutions to customizable multifunction integration, channel scaling, and 3D array formation were demonstrated.

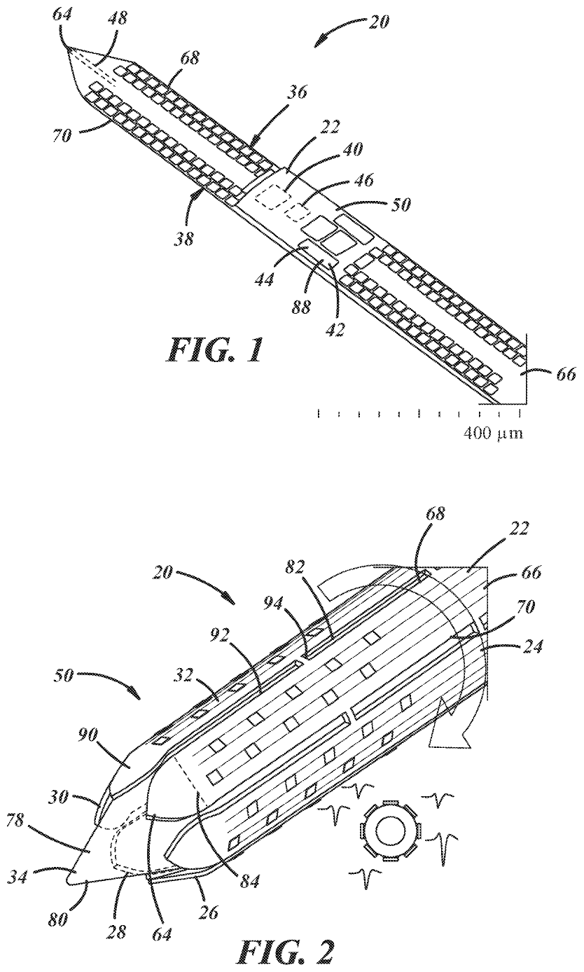

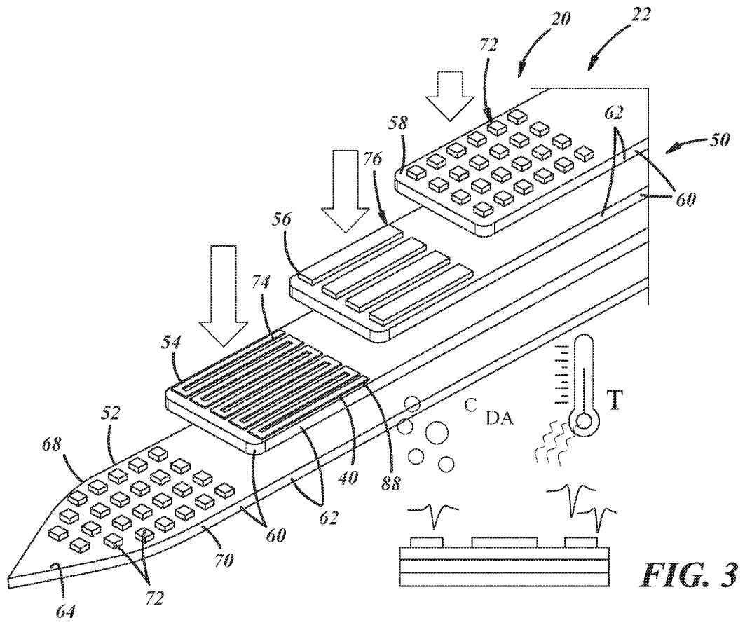

With reference to FIGS. 1-3, example probe assemblies 20 are shown. In FIGS. 1 and 3, there is shown a probe assembly 20 with a single probe shank 22. In FIG. 2, the probe assembly 20 has a plurality of probe shanks 22, 24, 26, 28, 30, 32 wrapped around a core region 34. The discussion herein is focused on the probe shank 22, but it should be understood that teachings relating to the probe shank 22 are applicable to other probe shanks, such as the probe shanks 24, 26, 28, 30, 32. Moreover, it is possible for the probe shanks 22, 24, 26, 28, 30, 32 to have varying functionalities, and they may not all be the same (e.g., one includes temperature sensing, a separate one includes neurotransmitter sensing, etc.). However, in the embodiments, particularly illustrated in FIG. 1, the probe assembly 20 includes a plurality of recording electrodes 36 and/or sensing electrodes 38, a temperature sensor 40, and a neurotransmitter sensor 42, which in this embodiment, is a dopamine sensing electrode 44. It's also possible to have one or more micro LEDs 46, chemical delivery channels 48, or other functional devices depending on the desired implementation. Each probe assembly 20 is an intracortical neural probe 50 configured to be inserted into neural tissue, although it is possible for the probe assembly 20 to be used in alternate implementations.

FIG. 3 shows one implementation of a probe shank 22 having a plurality of probe sub-units 52, 54, 56, 58. Each probe sub-unit 52, 54, 56, 58 is in essence its own separate probe, with the sub-units then being assembled together, as detailed further below. While a multi-layer implementation is advantageous, it is possible for the probe shank 22 to only be a single functional layer or sub-unit, depending on the desired implementation. Each of the probe sub-units 52, 54, 56, 58 has a flexible body 60 that serves as the main probe structure and shape. The flexible body is advantageously one or more polymer thin film layers 62, or more particularly, patterned polyimide thin film layers (e.g., PI-2610). The low Young's modulus and high yield strain material properties of polymer thin films allow them to be bent and folded around desired structures, while their low mass-density and large surface-to-volume ratio enable manipulation of them in liquid with minimal forces. The probe sub-units 52, 54, 56, 58 are assembled together, as detailed below, to create the probe shank 22 having an insertion end 64 configured to be inserted into neural tissue, a back end 66 configured to connect to one or more PCBs (possibly via a flexible cable), a first longitudinal side 68, and a second longitudinal side 70. The first and second longitudinal sides 68, 70 are the longest sides of the probe shank 22.

In the embodiment illustrated in FIG. 3, the probe sub-unit 52 has a plurality of recording electrodes 72. The probe sub-unit 54 has a thermal resistor 74 that is used as the temperature sensor 40. The probe sub-unit 56 has a plurality of chemical sensing electrodes 76, and the probe sub-unit 58 also has a plurality of recording electrodes 72. Each probe sub-unit 52, 54, 56, 58 has its own functionality, and are operable on their own as a distinct probe. Advantageously, the probe sub-units 52, 54, 56, 58 are laminated together to align each of the first and second longitudinal sides 68, 70 and create a multi-layer probe shank 22. However, as addressed above, it is possible in some implementations to only use one probe sub-unit as the probe shank.

Advantageously, as illustrated, self-assembly of up to four single shank flexible probe sub-units 52, 54, 56, 58 with sixty-four electrodes each (spanning 400 um) were demonstrated to create a 256-channel probe with a single shank 22, which is the highest known channel count for a single shank flexible probe. The width and spacing of the high-density interconnection traces were both 1 μm. The area of a single electrode 72 was 250 μm ². The electrodes 72 were defined at a high density with a 25-μm center-to-center distance between two adjacent electrodes for high spatial resolution. Each single unit signal was able to be recorded by over 8 electrodes in two columns. Up to 128-channel recordings were performed. The total number of simultaneous recording channels could be further increased by integrating application-specific integrated circuit (ASIC) chips. Multi-shank probes could be laminated together with the same approach to create 512-channel probe assemblies 20, for example. High-density interconnection metal traces were patterned at a pitch of 1-μm/1-μm or 1-μm/3-μm on the probe shank 22 to ensure miniaturized probe assembly 20 size (e.g., 70˜130 μm in width between the longitudinal sides 68, 70). The microelectrodes 72 were made with surface roughened Ti/Pt to improve the electrode-to-polyimide adhesion and reduce the impedance.

The chemical sensing electrodes 74 are advantageously dopamine sensing electrodes 44 having reduced-graphene oxide on a roughened platinum-based substrate 88. Dopamine sensing can be achieved by measuring currents generated by oxidation of dopamine. In order to detect dopamine effectively, the roughened Pt electrode surface 88 was modified with reduced-graphene oxide (rGO). The synergy of the roughened Pt surface and the reduced-graphene oxide modified electrode surface resulted in an efficient oxidation of dopamine which leads to the sensitive and selective quantitation of dopamine by direct oxidation of it at about 0.4 V (vs. Ag/AgCl 3M NaCl). The electrochemical properties of the reduced-graphene oxide (rGO) modified roughened Pt electrodes 88 were tested with electrochemical impedance spectrum (EIS), cyclic voltammetry (CV), and amperometry. A Nyquist plot obtained by EIS measurement before and after rGO modification showed that the electron transfer resistance was reduced from 385.9 k Ω to 143.0 k Ω. With rGO modification, the peak current of cyclic voltammogram increased from 114.1 nA to 334 nA. The dopamine sensor 44 sensitivity and selectivity were characterized by introducing dopamine and other interference chemicals with known concentration to PBS during amperometry measurement. The characterized sensitivity was 286±12 pA/μM with a linear range of 2.4 μM to 153.4 μM with and a detection limit of 28 nM. In the selectivity test, γ-aminobutyric acid (GABA) and uric acid (UA) induced negligible current change and amino acid (AA) induced slight (<10%) current increase.

As shown in FIGS. 2, 4, and 5, multiple probe shanks 22, 24, 26, 28, 30, 32 may be situated around a core region 34. In this embodiment, the probe shanks 22, 24, 26, 28, 30, 32 are circumferentially arranged around an optical fiber 78 that has a conically shaped insertion point 80. The optical fiber 78 is desirable, as it can be used for light transmission and optogenetic stimulation. However, in other implementations, the core region 34 could be a more standard insertion needle, or it could be an open recess or space, to cite a few examples.

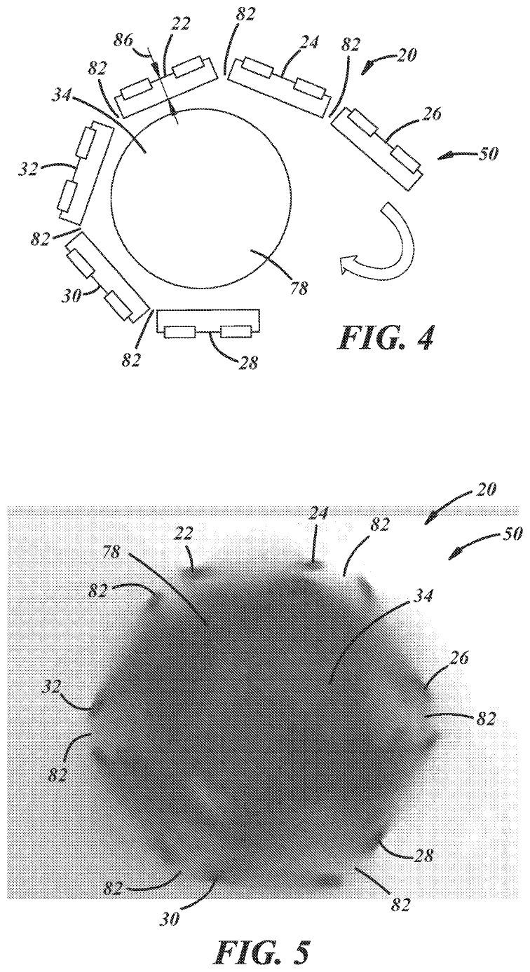

To facilitate capillary induced bending, as schematically illustrated in FIG. 4 and shown in the embodiments of FIG. 2 and FIG. 5, each probe shank 22, 24, 26, 28, 30, 32 includes one or more hinges 82, 84 that can help connect each shank or portions of each shank. The hinges 82, 84 extend into a thickness 86 of the probe shank, which allows for the flexible body 60 to further bend and conform at least partially around the core region 34. The hinge 82 extends longitudinally down along the probe shank 22 from the insertion end 64 toward the back end 66, and as shown in FIG. 2, it extends between a plurality of rows of electrodes 72. As schematically shown, the hinge 84 extends transversely across the probe shank 22 from the first longitudinal side 68 to the second longitudinal side 70. The transverse hinge 84 can be used to delineate the conically shaped insertion point 80 and thus delimits a conical insertion tip 90 on the probe assembly 20.

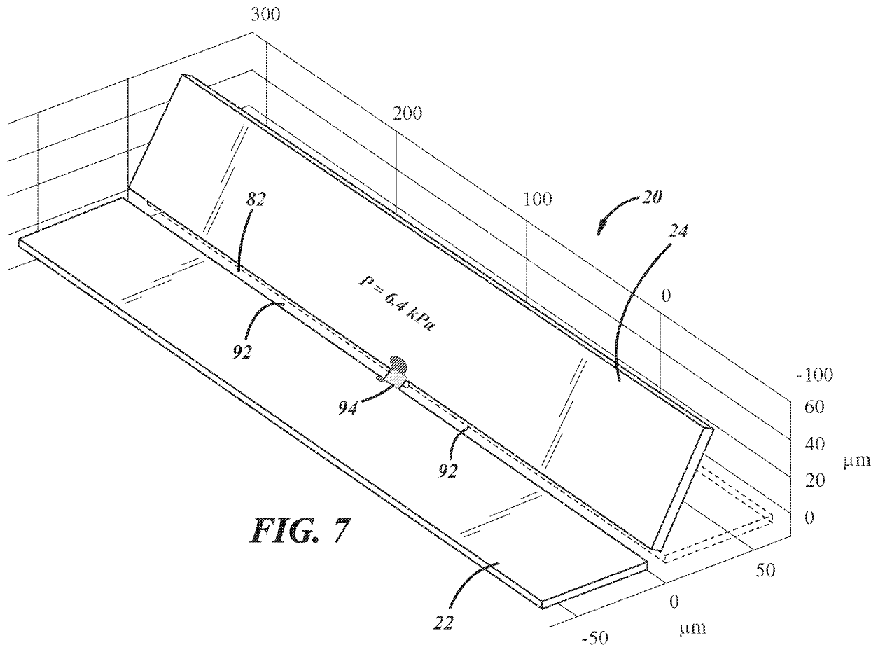

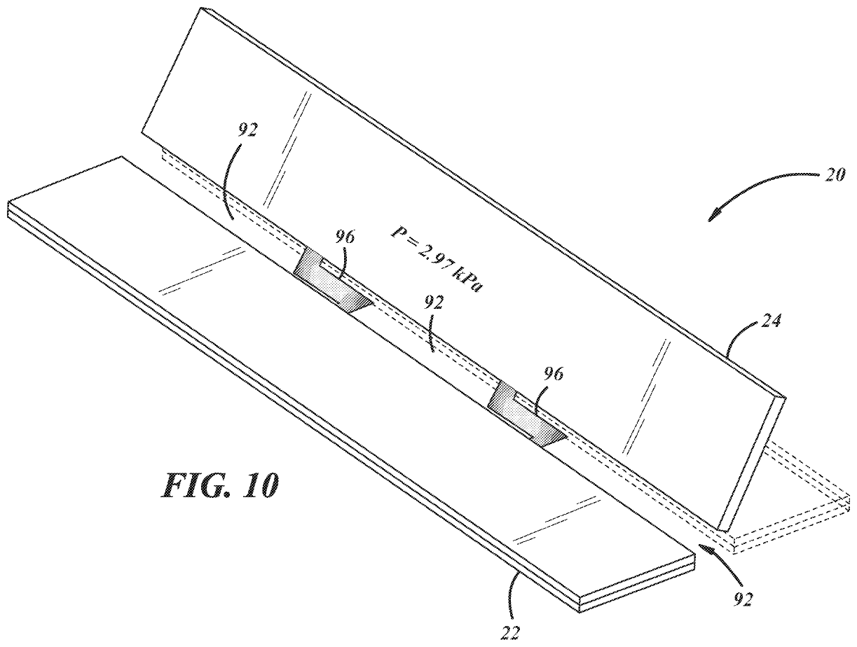

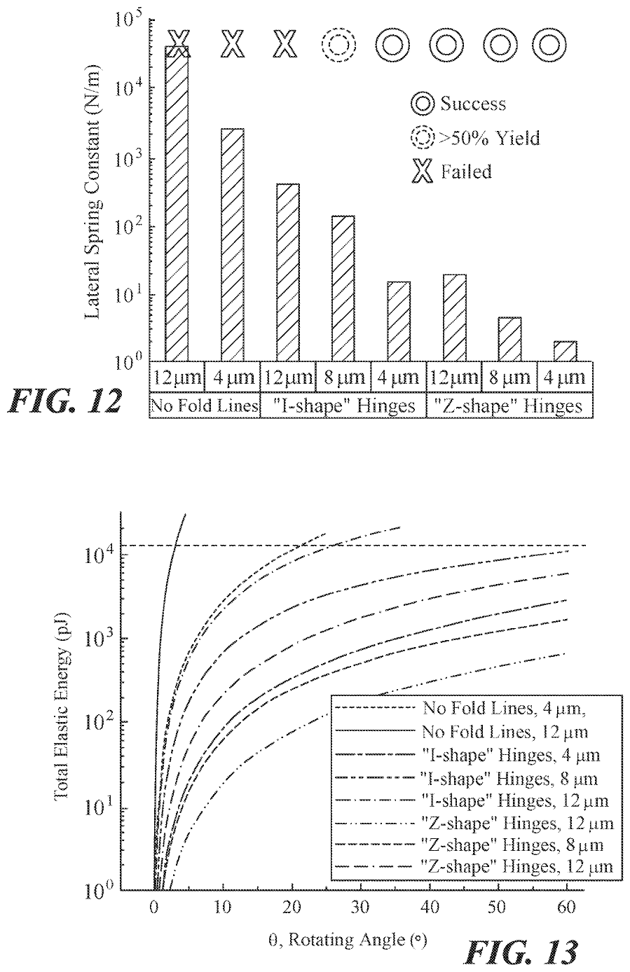

In the embodiment shown in FIGS. 2-11, and with particular reference to the embodiment of FIGS. 2 and 6-8, the hinge 82 comprises multiple fold lines 92 that extend entirely through the thickness 86 of the probe shank 22 and an I-shaped structure 94 at the midpoint of the longitudinal extent. More I-shaped structures 94 can be located to delineate additional fold lines 92, but this may depend on the desired implementation, length of the probe shank 22, necessary degree of bending (see angle θ), etc. For various probe shank thicknesses 86, FIGS. 12 and 13 show advantageous lateral spring constant values and elastic energy for a given rotating or bending angle θ, respectively.

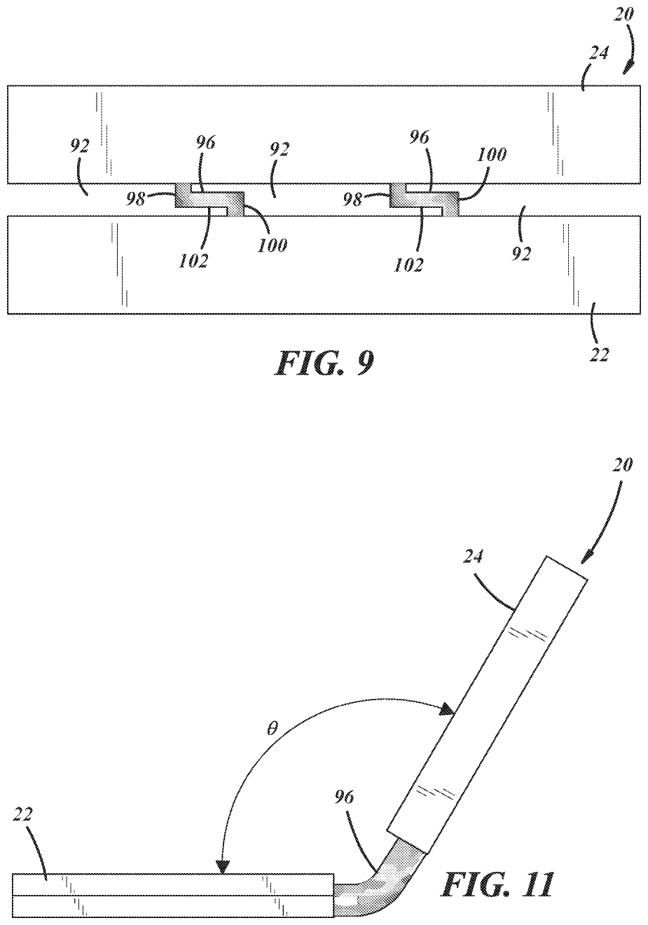

In the embodiment of FIGS. 9-11, the hinge 82 includes multiple fold lines 92 delineated by Z-shaped structures 96. The Z-shaped structures 96 have two laterally extending portions 98, 100 that connect the probe shanks 22, 24 and a longitudinally extending portion 102 that is separate from each of the probe shanks 22, 24 and connected to each probe shank by the laterally extending portions 98, 100. The Z-shaped structures 96 were found to provide greater flexibility than alternately configured hinges 82.

The I-shaped and Z-shaped structures 94, 96 are patterned polyimide and the fold lines 92 are etched in to create the low-bending stiffness structures and promote foldability. The fold lines 92 in the illustrated embodiments extend 100% into the thickness 86 of the probe shank 22, and at each of the I-shaped and Z-shaped structures 94, 96, the thickness can be etched or otherwise decreased to further improve bending ability. This thickness decrease at the I-shaped and Z-shaped structures 94, 96 may be about 50% in one embodiment, or at an operable value between 0-90% into the thickness. With the elastic hinges 82, and more particularly I-shaped and Z-shaped structures 94, 96, and the fold lines 92 or etched gaps, single or double-layer origami probe shanks, each probe shank having a plurality of probe sub-units can be folded to an angle θ of about 60° given an inward pressure less than the estimated capillary induced pressure (e.g., 15 kPa), the hinge 82 being incorporated on each probe sub-unit.

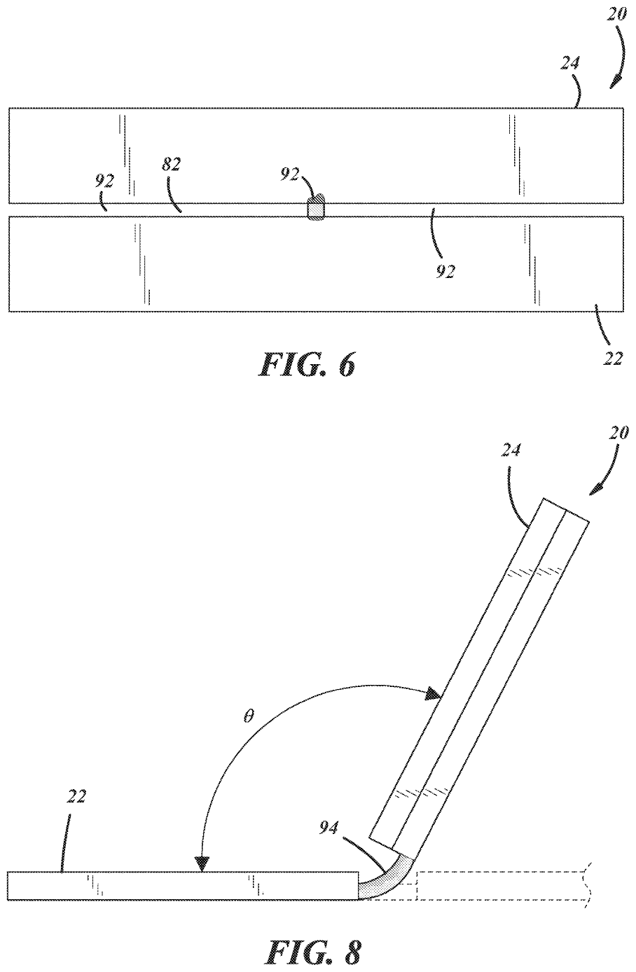

The COMSOL simulation shown in FIGS. 6-11 was used to investigate different fold-line designs of the origami neural probe assembly 20. In the model, a pair of single-or double-layer shanks (60 μm ×400 μm) are connected with thinned elastic hinges modelled using polyimide material (Young's Modulus: 8.5GPa). A periodic boundary condition was applied on the start and end surfaces (longitudinal direction) to simulate the full probe shank. A fixed boundary constraint was applied to one shank. A uniform pressure was applied onto the other shank to mimic the capillary force. A range of the boundary load was simulated by parametric sweep. The displacement, bending angle, first principal strain and total elastic energy were recorded at each step. The I-shaped and Z-shaped hinge structures 94, 96 and the fold lines 92 appeared sufficient to impart the requisite bending capability necessary to create a 360° recording probe assembly 20.

To create the structure shown in FIGS. 2, 4, and 5, controllable capillary assisted wrapping of the origami neural probe assembly 20 was made possible by the longitudinal fold lines 92 and hinges 82. The fold lines 92 were defined by a two-step reactive ion etching. The first etching step completely removed most of the polyimide flexible body 60 and left the elastic hinges 82/fold lines 92 having I-shaped structures 94 connecting the probe shanks 22, 24, 26, 28, 30, 32. The second etching thinned the thickness of the hinges 82 to half of their total thickness at the I-shaped structures 94. The hinges 82 allowed the probe assembly 20 to hold its shape and be wrapped in a controllable manner before and during self-assembly. 3D origami probes of different diameters could be created by adjusting the design and total width of the assembly 20 with respect to a specified fiber 78 or core region 34 diameter. As a demonstration, 3D origami probes of three different diameters, 125-μm, 105-μm, and 80-μm, were created. Double-sided probe assemblies 20 were created by reducing the number of hinges 82 to one and without using the optical fiber 78 for the core region 34.

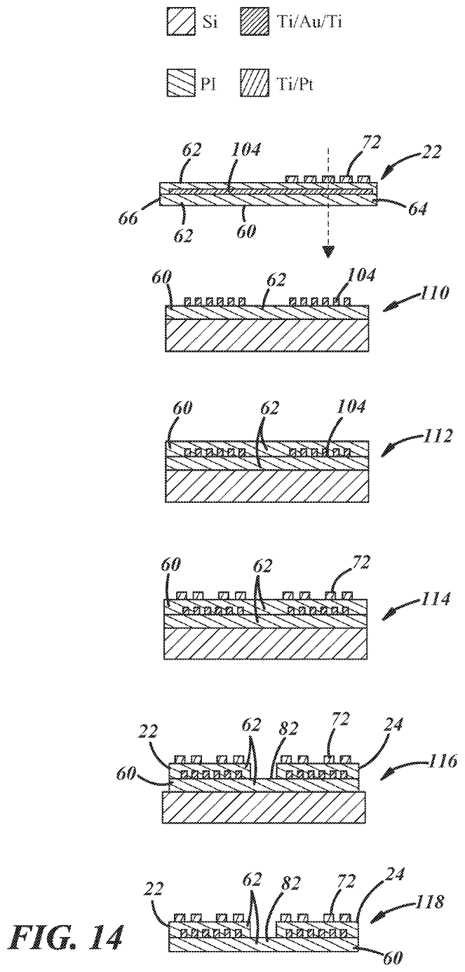

FIG. 14 schematically illustrates a method of manufacturing a probe shank 22 for a probe assembly having a hinge 82, recording electrodes 72, and flexible cable 104. Flexible neural probe assemblies were microfabricated using MEMS technologies following a thin-film polyimide device fabrication process. First, a Cr/Au/Cr sacrificial layer with 100/500/500 Å thickness was evaporated over a 4-inch silicon wafer coated with a layer of 500-nm silicon oxide. Polyimide was span over the sacrificial layer at 3000 rpm. A soft baking at 115° C. for 10 minutes was applied to remove the solvent. The polymerization process was completed in a vacuum oven by ramping the temperature to 350° C. and holding for 1 hour. The average thickness of a single polyimide after polymerization was 2 μm. Second, a layer of lift-off resist was span on at 2000 rpm and baked at 200° C. for 5 minutes. On top of it, a layer of photoresist was span on at 2000 rpm and baked at 115° C. for 90 seconds. The 2 μm pitch patterns was created using a projective lithography tool. After plasma descum for 60 seconds, Ti/Au/Ti with a thickness of 100/1000/100 Å was evaporated and lift-off patterned by fully dissolving the photoresist and lift-off resist in Remover PG (see e.g., step 110). After dehydration bake, the wafer was plasma activated with oxygen plasma. The polyimide coating and curing process was repeated (see e.g., step 112), and material for recording electrodes 72 was patterned (see e.g., step 114).

For forming the hinges 82, aluminum oxide (Al₂O₃) with 200 Å thickness was deposited at 150° C. by atomic layer deposition as a polyimide etching hard mask. A 3 μm SPR 220 3.0 photoresist was span coated and patterned to define the outline and fold-lines 92 of the flexible probe shank 22. Diluted HF acid was used to etch the unmasked Al₂O₃ to expose the polyimide underneath. The polyimide without masking was then etched away by reactive ion etching. The hinge 82 can be formed with a timed etch or by introducing an etch-stop layer (see e.g., step 116). With a similar process, contact vials exposing 3-μm by 3-μm windows on the encapsulation polyimide layer were etched. For the origami probe assembly 20, the connecting elastic hinges 82 were further thinned to 2 μm by timed etching. After polyimide etching, the Al₂O₃ hard mask was removed. A layer of sub-nanometer Ti was sputtered using a metal sputter deposition tool to form a non-continuous etching hard mask. The surface of the top polyimide layer was roughened by reactive ion etching through the non-continuous Ti layer. Ti/Pt with a thickness of 100/1000 Å was sputtered and lift-off patterned with a similar lithography process to form the electrodes 72 (see e.g., step 114 above). Last, the sacrificial layer was removed with Cr etchant. The flexible probe shanks 22, 24 were then released from the substrate (see e.g., step 118).

To manufacture the probe assembly 20 shown in FIGS. 2, 4, and 5, a capillary induced bending method is used. The probe shanks 22, 24, 26, 28, 30, 32 were first immersed in isopropyl alcohol (IPA), which has a lower surface tension coefficient compared to water. With the specially designed elastic hinges 82, the probes remained flat during and after being removed from the IPA. After being fully dried, the probe assembly 20 was transferred to a temporary holder with its shank free standing. Next, an optical fiber 78 was coarsely aligned and attached at the end of the flexible cable 104 with its tip protruding. Finally, the free-standing probe shank and the fiber tip were immersed in and slowly (˜1 mm/s) pulled out of DI water. During the pulling, the origami probe assembly 20 was fully wrapped over the fiber 78 by capillary force. The self-assembly lamination approach was successfully applied to the 3D origami probe assemblies 20 as well for channel count scaling and multifunction integration.

To obtain successful wrapping, different types of fold line 92 designs were modeled and experimentally validated with different probe shank 22 thicknesses 86 (see e.g., above and FIGS. 6-13). During the wrapping process, the elastic energy of the hinges 82 increased while the surface energy decreased. An inward pressure on the shank 22 induced by capillary force increased as the liquid exited and the gap between the probe shank 22 and fiber 78 closed. Based on the model geometry (e.g., 60 μm width between fold-lines 92 and 400 μm length between two hinges 82), the total surface energy reduction and maximum inward pressure were roughly estimated as 13.9 nJ and 15 kPa, respectively. The net system energy change combined with the predicted inward pressure for 60° hinge 82 bending could indicate whether the wrapping could be achieved. The experiment results matched the prediction. After wrapping, the origami probe assembly 20 attached to the surface of the fiber 78 in a hexagon shape. This was because the adhesion between the fiber 78 and probe shank surfaces was not strong enough to achieve a conformal attachment. We also verified that the 3D origami probe shanks did not detach from the fibers 78 during insertion. The tests were performed by inserting and imaging the probe assemblies 20 into agarose gel (0.6 wt. %), which mimicked brain tissue consistency. The tips of the probe assemblies 20 were imaged perpendicularly to the insertion direction using a transmission microscope. Without any adhesive materials applied, no delamination or unwrapping was observed during three insertion and retrieval trials. In each trial, the probe assembly 20 was inserted into the gel for over 8 mm deep.

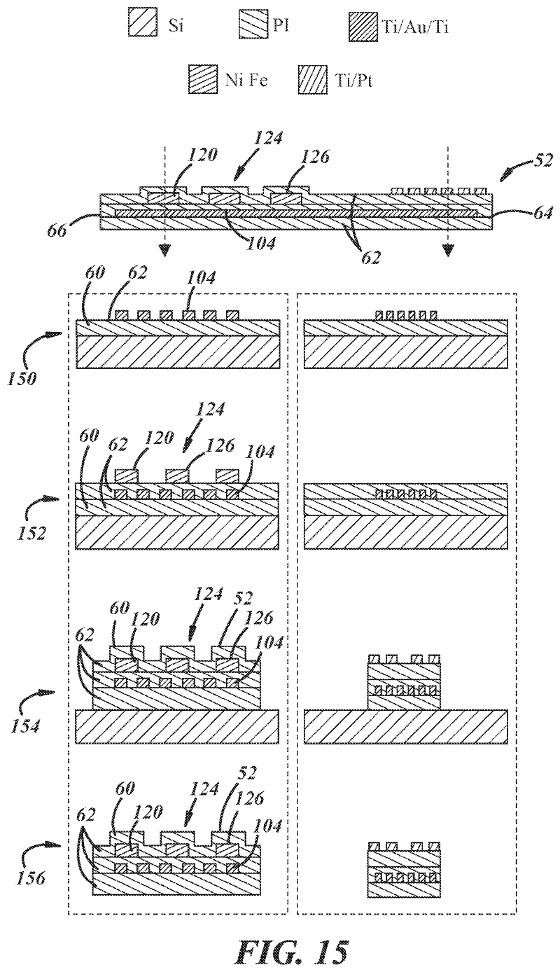

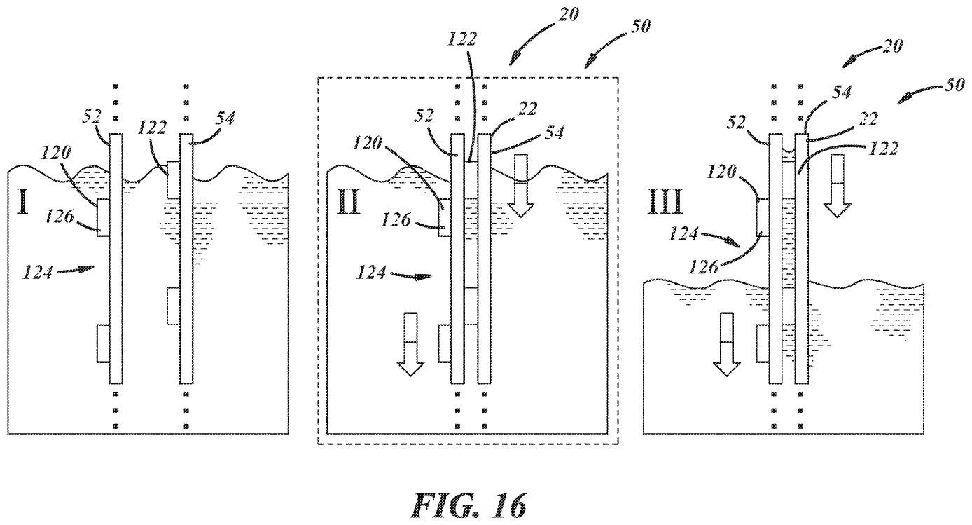

FIGS. 15 and 16 schematically illustrate a cross-section view of one embodiment of a probe sub-unit 52 used in the probe shank 22, and FIG. 16 more particularly shows the assembly of the first probe sub-unit 52 to a second probe sub-unit 54 to form the multi-layer probe shank 22 (e.g., that shown in FIGS. 1-3). In accordance with this lamination method, at least a first magnet 120 on the probe sub-unit 52 and a second magnet 122 on the second probe sub-unit 54 are coupled to form the probe shank 22 and probe assembly 20. Each magnet 120 is an array of micromagnets 124 that are schematically illustrated in the figures. More particularly, a seed layer and a Ni80Fe20 magnet layer 126 were patterned to form the array of micromagnets 124. Preferably, a magnetic material which is compatible with microfabrication is used, and other materials besides Ni80Fe20 are possible, such as electroplated Ni, Co, Fe alloys, to cite a few examples.

A simplified schematic microfabrication process is illustrated in FIG. 15 for manufacturing one of the probe sub-units 52 shown in FIG. 1. For the probe sub-unit 54 (see FIG. 3), the temperature sensor 40, which is a thermal resistor 74, was encapsulated by polyimide. The thermal resistor 74 is made of a 100/1000/100 nm Ti/Au/Ti trace in a meander pattern. Four interconnection lines were connected to the thermal resistor 74 to determine its resistance using four-point measurement. The temperature coefficient of resistance was 0.19±0.014% (N=4). The roughened electrode was created by sputtering and patterning Ti/Pt over the roughened polyimide surface. The surface morphology was visualized by SEM imaging from a 45° angle. At 1 kHz, the electrochemical impedance was 474.3±34 kΩ (N=16 electrodes) measured with an LCR-meter in phosphate-buffered saline (PBS). The roughening processes reduced the electrode impedance by around three-fold. The DC magnetic hysteresis curve for the permalloy micromagnet was measured using a vibrating sample magnetometer (VSM) system. The resulting M-H curve was diamagnetically corrected, indicating a magnetic saturation of ˜813 kA/m.

Ti/Au with a thickness of 100/500 Å was evaporated and lift-off patterned with a similar lithography process to form the seed layer (see e.g., step 150, FIG. 15). Next, a thicker (5 μm) layer of photoresist was span on and patterned. After plasma descum, Ni80Fe20 micro-magnets with a thickness of 4 μm were electroplated with DC current and a current density of 15 mA/cm² for 15 mins (see e.g., step 152). A 10-cm by 10-cm meshed Ni plate was used as the electroplating anode. The wafer and the Ni anode were immersed in the electroplating bath and separated by approximately 5 cm in a 2000-mL size glass beaker. Continuous stirring was applied with a magnetic stirrer. The micromagnet arrays 124 were about 100-μm by 500-μm and embedded in polyimide polymer thin film layers 62 on the flexible cable 104 (see e.g., step 154). No magnets were embedded on the electrode portion of the sub-unit. The magnet 120/magnet array 124/magnet layer 126 should be large enough so that the magnetic attraction is large enough when assembling the probe sub-nits, and they should be electrically insulated or embedded. Embedding the magnet array 124 in polyimide so as to at least partially shield them from the electrodes and other probe sub-unit components helps with anti-corrosiveness, preventing delamination, and fabrication compatibility. The probe sub-unit 52 can be released from the substrate (see e.g., step 156), and then magnetically laminated with one or more other probe sub-units, as shown schematically in FIG. 16.

During the self-assembly process schematically illustrated in FIG. 16, the flexible cables of the two probe sub-units 52, 54 were self-aligned and attached together in isopropyl alcohol (IPA) by magnetic force. A pair of the magnet embedded probe sub-units 52, 54 were placed in the same orientation in IPA. An external magnetic field in parallel to the probe sub-units 52, 54 was applied to magnetize the magnets 120, 122 to saturation. Next, the IPA container was shaken gently in the direction perpendicular to the probes with an approximate travel distance of 5-mm, so that the probes move to the minimum energy position. The attraction force between the magnet arrays 120, 122 on the two probe sub-units 52, 54 further adhered them together. Coarse alignment was achieved as the magnets 120, 122 were attached head-to-tail. The probe sub-units 52,54 were aligned and attached together by capillary force when the probe sub-units were next pulling out of IPA.

The self-assembly approach schematically illustrated in FIG. 16 could also be applied to laminate the origami probe assemblies and multi-shank flexible probe assemblies (see e.g., FIG. 2). After a MULTI probe assembly 20 was assembled and fully dried, it did not separate when being re-immersed in IPA for a short time (less than one hour, approximately). More probe shanks or probe sub-units could be added by repeating the self-assembly process using an assembled MULTI and one additional probe sub-unit each time, with each probe sub-unit advantageously having an embedded magnet. The three probe sub-unit laminated multifunctional MULTIs 20 with 128 recording channels, one temperature sensor 40, and four dopamine sensing electrodes 44 were prepared this way. The sub-unit of each flexible probe shank has a thickness of 5 μm. Therefore, with three probe sub-units laminated together, the total thickness is 15 μm, in this embodiment. A 128-channel amplifier board was connected to the PCB for signal acquisition. The entire backend (with the amplifier board connected) has a weight of 5.8 grams and a size of 24 mm by 39 mm in this implementation.

Another potential manufacturing step includes manufacture of the dopamine sensing electrode 44. In one embodiment, roughened Pt electrode surfaces 88 can be pre-conditioned by cyclic potential scanning (−1.0˜+1.0V at a scan rate 100 m V/s) in PBS using a with a conventional three-electrode configuration. Specifically, the working electrodes were the roughened Pt electrode (40 μm×50 μm). The counter electrode was a platinum wire electrode. The electrical potentials were relative to an Ag/AgCl (in 3 M NaCl) reference electrode. After conditioning, the electrodes 44 were rinsed with DI wafer. Graphene oxide was dissolved in the DI water using an ultrasonic bath for 2 hours. Next, the electrodes 44 were modified with reduced rGO by cyclic potential scanning (10 cycles, −1.0V˜+1.0V, scan rate 50 mV/s) in a mixture of rGO (3 mg / mL) and Na₂SO₄ (0.1 M) solution. The rGO modified electrodes 44 were air-dried for 30 minutes at room temperature.

Amperometry and cyclic voltammetry measurement were performed using a potentiostat. The in vitro experiments were carried out with the three-electrode configuration in a 15-mL electrochemical glass cell. A cyclic voltammetry and Nyquist plot was measured in K₃Fe(CN)₆/K₄Fe(CN)₆ (1:1, v/v, 5.0 mM) solution dissolved in PBS (50 mM) at pH 7.0. Electrochemical impedance spectroscopy (EIS) was characterized in PBS. In vitro dopamine concentration measurements by amperometry experiments were performed in PBS with constant stirring. A constant potential of +0.4 V (vs. Ag/AgCl, 3M NaCl) was applied to the working electrode 44 (rGO modified roughened Pt electrode 88) and the background current was allowed to decay to a steady state before aliquots of standard solution of dopamine were added to the electrochemical cell.

Neural recording (128 channel), temperature monitoring, and dopamine concentration sensing using the multifunctional MULTI probe assembly 20 of FIG. 1 were demonstrated in acute studies. Although it allowed for three functions to be performed simultaneously, to simplify the experiments, the effects of local brain temperature and dopamine concentration on neural activities were examined separately. Urethane anesthesia was used in both experiments. The MULTI probe shank 22 was implanted targeting simultaneous recording in cortex and hippocampus regions. The probe shank 22 was mounted on the surface of a 200-μm diameter stainless-steel syringe needle coated with PEG. The syringe was fixed on a linear manipulator for insertion. The results showed operable functionality of the probe assembly 20.

The MULTI probe assembly 20 created by magnetic and capillary hybrid self-assembly is highly customizable and scalable. Flexible neural probe shanks 22 with different electrode array configuration or functional components could be fabricated and integrated in a modularized way. The probe assemblies 20 demonstrated 128-channel neural recording simultaneously with local tissue temperature or dopamine concentration monitoring. The probe assemblies 20 benefited from the high channel count, and a large population of single units could be identified allowing for different neuronal behaviors to be distinguished. The customized span of the electrode arrays enabled simultaneous recording of different brain regions. In additional to the presented MULTI probe assembly 20, flexible probes with other functions such as a waveguide, micro-LEDs, and drug delivery channels could be produced and integrated similarly. For the probe assemblies 20 designed herein, the distance between two micromagnet arrays 124 (first and second magnets 120, 122) was 2 mm. As the length of each micro-magnet 120, 122 is 500 μm, reliable self-assembly of four probe sub-units 52, 54, 56, 58 was achieved. To further laminate more probe sub-units, a shorter magnet or wider spacing may be used to accommodate the reduction in magnetic attraction force.

Additionally, the 3D origami probe assembly 20 having one or more hinges 82, 84 is an advantageous tool for acute brain mapping (see e.g., FIG. 2). It is by far the highest density cylindrical 3D probe with real 360° recording and neuron locating capability. With a combination of the self-assembled lamination approach and the 3D origami probe assembly 20 design, further scaling to 128 and more channels could be achieved. In some embodiments, softer and biocompatible polymer fibers or a hollow cylindrical sheath can be used to replace the optical fibers. With regard to the cross-sectional shape, a more circular outline could be achieved by coating additional adhesive material over the fiber 78 or increasing the number of the probe shanks. The outline could potentially hold a circular shape naturally with compression from tissues pressing the probe shanks 22, 24, 26, 28, 30, 32 against the fiber 78. For applications where a rotational symmetric full 360° field of view is not desired, the double-sided probes can be a good alternative. The miniaturized cross-section size (e.g., 70˜130 μm wide and 8 μm thick) could make the double-sided probe more suitable for chronic implantation and small animal applications.

For acute recording with the 3D origami probe assembly 20, the optical fiber 78 (e.g., 105 μm diameter with a tapered tip) was first mounted onto a PCB holder which fixed the PCB onto the linear manipulator for insertion. The position of the monolithic-fabricated flexible cable 104 was adjusted to align the planar origami probe shanks 22, 24, 26, 28, 30, 32 with the optical fiber 78. The probe shanks and the optical fiber were then self-assembled in DI water and ready for insertion. To further secure the probe shanks 22, 24, 26, 28, 30, 32 to the fiber 78, PEG coating can be applied. The cone-tip optical fiber 78 used was sharp and rigid so that no additional insertion shuttle was needed.

Single unit recording from neurons 360° surrounding the 3D origami probe assembly 20 depicted in FIG. 2 was also successfully demonstrated. Angular heterogeneity was easily seen in both the single unit activities and the local field potentials as demonstrated by the measured waveforms, spike raster plot, and the representative single units. Expanding the field of view to 360° contributed to identifying more completed neural connecting circuits. In comparison, a single-sided probe may leave part of the connecting circuits in its blind spots.

It is to be understood that the foregoing description is of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “e.g.,” “for instance,” and “such as,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation. In addition, the term “and/or” is to be construed as an inclusive OR. Therefore, for example, the phrase “A, B, and/or C” is to be interpreted as covering all the following: “A”; “B”; “C”; “A and B”; “A and C”; “B and C”; and “A, B, and C.”

1. A probe assembly, comprising:
a probe shank;
a hinge extending into a thickness of the probe shank; and
a core region, wherein the probe shank has a bend at the hinge to conform at least partially around the core region.

2. The probe assembly of claim 1, wherein the hinge extends longitudinally down the probe shank from an insertion end towards a back end.

3. The probe assembly of claim 2, wherein the hinge extends between a plurality of rows of electrodes.

4. The probe assembly of claim 1, wherein the hinge extends transversely across the probe shank from a first longitudinal side toward a second longitudinal side.

5. The probe assembly of claim 4, wherein the hinge delineates a conical insertion tip.

6. The probe assembly of claim 1, wherein the hinge has an I-shaped structure or a Z-shaped structure.

7. The probe assembly of claim 6, wherein the thickness of the probe shank is etched at the I-shaped structure or the Z-shaped structure.

8. The probe assembly of claim 1, wherein the probe shank includes a dopamine sensing electrode comprising reduced-graphene oxide on a roughened platinum-based substrate.

9. The probe assembly of claim 1, wherein the probe shank comprises a first probe sub-unit and a second probe-sub unit, wherein the first probe sub-unit has the hinge and the second probe-sub unit has a hinge.

10. The probe assembly of claim 9, wherein the first probe sub-unit and the second probe sub-unit are laminated together with magnets.

11. The probe assembly of claim 1, wherein the hinge comprises a plurality of fold lines that extend fully through the thickness.

12. The probe assembly of claim 1, wherein the core region is an optical fiber.

13. The probe assembly of claim 12, comprising a plurality of probe shanks that are circumferentially arranged around the optical fiber, and wherein an insertion end of each probe shank is conformally arranged around a conically shaped insertion point of the optical fiber.

14. A method of manufacturing a probe assembly, comprising the step of:
bending a portion of a probe shank around a core region at a hinge.

15. The method of claim 14, wherein the bending step is capillary induced.

16. A probe assembly, comprising:
a probe shank having a first probe sub-unit and a second probe sub-unit, wherein the first probe sub-unit has a first magnet, and the second probe sub-unit has a second magnet, wherein either the first probe sub-unit or the second probe sub-unit has a plurality of electrodes, and wherein the first magnet, the second magnet, or both the first magnet and the second magnet are at least partially shielded from the electrodes.

17. The probe assembly of claim 16, wherein each of the first magnet and the second magnet are encapsulated in polyimide.

18. The probe assembly of claim 16, wherein the first magnet and the second magnet are each an array of micromagnets.

19. A method of manufacturing the probe assembly of claim 12, comprising the step of:
adhering the first probe sub-unit and the second probe sub-unit with the first magnet or the second magnet.

20. The method of claim 19, wherein the adhering step is capillary induced.

1. A probe assembly, comprising:
a probe shank;
a hinge extending into a thickness of the probe shank; and
a core region, wherein the probe shank has a bend at the hinge to conform at least partially around the core region.

2. The probe assembly of claim 1, wherein the hinge extends longitudinally down the probe shank from an insertion end towards a back end.

3. The probe assembly of claim 2, wherein the hinge extends between a plurality of rows of electrodes.

4. The probe assembly of claim 1, wherein the hinge extends transversely across the probe shank from a first longitudinal side toward a second longitudinal side.

5. The probe assembly of claim 4, wherein the hinge delineates a conical insertion tip.

6. The probe assembly of claim 1, wherein the hinge has an I-shaped structure or a Z-shaped structure.

7. The probe assembly of claim 6, wherein the thickness of the probe shank is etched at the I-shaped structure or the Z-shaped structure.

8. The probe assembly of claim 1, wherein the probe shank includes a dopamine sensing electrode comprising reduced-graphene oxide on a roughened platinum-based substrate.

9. The probe assembly of claim 1, wherein the probe shank comprises a first probe sub-unit and a second probe-sub unit, wherein the first probe sub-unit has the hinge and the second probe-sub unit has a hinge.

10. The probe assembly of claim 9, wherein the first probe sub-unit and the second probe sub-unit are laminated together with magnets.

11. The probe assembly of claim 1, wherein the hinge comprises a plurality of fold lines that extend fully through the thickness.

12. The probe assembly of claim 1, wherein the core region is an optical fiber.

13. The probe assembly of claim 12, comprising a plurality of probe shanks that are circumferentially arranged around the optical fiber, and wherein an insertion end of each probe shank is conformally arranged around a conically shaped insertion point of the optical fiber.

14. A method of manufacturing a probe assembly, comprising the step of:
bending a portion of a probe shank around a core region at a hinge.

15. The method of claim 14, wherein the bending step is capillary induced.

16. A probe assembly, comprising:
a probe shank having a first probe sub-unit and a second probe sub-unit, wherein the first probe sub-unit has a first magnet, and the second probe sub-unit has a second magnet, wherein either the first probe sub-unit or the second probe sub-unit has a plurality of electrodes, and wherein the first magnet, the second magnet, or both the first magnet and the second magnet are at least partially shielded from the electrodes.

17. The probe assembly of claim 16, wherein each of the first magnet and the second magnet are encapsulated in polyimide.

18. The probe assembly of claim 16, wherein the first magnet and the second magnet are each an array of micromagnets.

19. A method of manufacturing the probe assembly of claim 12, comprising the step of:
adhering the first probe sub-unit and the second probe sub-unit with the first magnet or the second magnet.

20. The method of claim 19, wherein the adhering step is capillary induced.
